(19) United States
(12) Patent Application Publication
(10) Pub. No.: US 2008/0242934 A1
(43) Pub. Date: Oct. 2, 2008

(54) ENDOSCOPIC SUCTION DEVICE FOR MUCOSECTOMY

(75) Inventors: GREGORY J. SKERVEN, Kernersville, NC (US); John A. Karpiel, Winston-Salem, NC (US)

Correspondence Address: BRINKS HOFER GILSON & LIONE/CHICAGO/COOK, PO BOX 10395, CHICAGO, IL, 60610, US

(73) Assignee: WILSON-COOK MEDICAL INC., WINSTON-SALEM, NC (US)

(21) Appl. No.: 12/056,639

(22) Filed: Mar. 27, 2008

Related U.S. Application Data
(60) Provisional application No. 60/920,829, filed on Mar. 29, 2007.

(51) Int. Cl. A61B 1/015

(52) U.S. Cl. 600/156

(57) ABSTRACT

An endoscopic suction device of an endoscope for mucosectomy is disclosed. The device comprises a connecting base having an open end attachable to the distal end of the endoscope. The device further comprises a distal tip extending from the connecting base and having a closed distal end. The distal tip comprises a suction chamber formed therein and in fluid communication with the open end. The suction chamber has a suction opening formed laterally therethrough for suctioning lesions during mucosectomy.

### CROSS-REFERENCE TO RELATED APPLICATIONS

This application claims the benefit of U.S. Provisional Application Ser. No. 60/920,829, filed on Mar. 29, 2007, entitled “ENDOSCOPIC SUCTION DEVICE FOR MUCOSECTOMY,” the entire contents of which are incorporated herein by reference.

### BACKGROUND OF THE INVENTION

The present invention relates to endoscopic suction devices and apparatus for medical procedures involving endoscopic procedures.

The treatment of tissue encompasses a variety of techniques such as electrocauterization, heat therapy, resection (removal of tissue), and sclerotherapy (the injection of medicine into target tissue). These treatment techniques usually involve the passing of medical instruments through the operating channel of the endoscope. The endoscope permits minimally invasive access, as well as visualization and suction aids.

Another technique that frequently utilizes the operating channel of the endoscope is ligation, which involves applying a band or ligature around a vessel or portion of tissue, thereby cutting off blood or fluid flow and causing the tissue to necrose and separate from adjacent healthy tissue. Ligation is widely used to treat a number of medical tissue conditions, including, but not limited to, hemorrhoids, polyps, ballooning varices, and other types of lesions, including those that are cancerous. Typically, ligators are also used with a suction or vacuum means to draw the tissue into the distal tip, whereby the band is deployed over the base of the diseased tissue to cut off blood flow. The ligating device is typically activated by retracting a line (string, wire, or cable) that is attached to the ligator at the distal end of an endoscope and is threaded through the operating channel of the endoscope to the proximal end of the instrument. The ligator can be activated by mechanically pulling the activating line by means of a hand-operated reel or trigger, or a motor drive mechanism. Various other ligating devices use cooperating inner and outer members that slide the individual bands by pushing or pulling them from the tip of the inner or outer member, the bands being preloaded onto the inner or outer member prior to deployment.

To prevent having to withdraw the instrument from the patient, reload, and reintroduce it for treating additional tissue or vessels, devices have been developed capable of sequentially delivering multiple bands that are preloaded, thus shortening the procedure time and improving patient comfort. Multiple band ligating devices include designs that individually tether or otherwise secure the bands to the dispenser and then release them sequentially as needed, often by use of one or more strings extending to the proximal end.

For example, during mucosectomy, the excision of a mucosa, the clinician faces challenges in removing mucosa merely due to the design of the distal end of a typical endoscope. More specifically, the suction port formed on the distal end of the endoscope may at times cause challenges, e.g., visual obstruction when a polypectomy snare is used to position around a lesion for removal thereof.

Thus, it is desirable to provide an endoscopic suction device that is compatible with an endoscope and that provides reduced visual obstruction during mucosectomy.

### BRIEF SUMMARY OF THE INVENTION

The present invention generally provides an endoscopic suction device that is compatible with an endoscope for endoscopic mucosal resection (EMR). Embodiments of the present invention provide a device to allow a more simplified way of mucosectomy, especially when a polypectomy snare is used therewith. A device allows for a relatively easier way of removing a lesion during mucosectomy.

In one embodiment, the device comprises a connecting base having an open end attachable to the distal end of the endoscope. The device further comprises a distal tip extending from the connecting base and having a closed distal end. The distal tip comprises a suction chamber formed therein and in fluid communication with the open end. The suction chamber has a suction opening formed laterally therethrough for suctioning lesions during mucosectomy.

In another embodiment, the present invention provides an endoscope apparatus. The apparatus comprises the endoscopic suction device and an endoscopic assembly for endoscopy. The apparatus comprises an insertion tube having a plurality of channels through which endoscopic parts may be disposed. The apparatus further comprises a control system in mechanical and fluid communication with the insertion tube. The control system is configured to control at least one of the endoscopic parts.

In another example, the present invention provides a method of mucosectomy of mucosal tissue of a patient. The method comprises disposing a polypectomy snare distally through a working channel of the endoscope having a distal end and advancing the snare through the distal end to the endoscopic suction device attached to the distal end. The method further comprises opening the snare adjacent the suction opening to receive the mucosal tissue and suctioning the mucosal tissue through the suction opening. The method further comprises receiving the mucosal tissue with the snare.

Further objects, features, and advantages of the present invention will become apparent from consideration of the following description and the appended claims when taken in connection with the accompanying drawings.

### BRIEF DESCRIPTION OF THE DRAWINGS

FIG. 1 is side view of an endoscopic suction device in accordance with one embodiment of the present invention;

FIG. 2 is a perspective view of a flexible endoscopic apparatus comprising the endoscopic suction device in accordance with one embodiment of the present invention;

FIG. 3 is an elevated view of a distal tip of the endoscope and the endoscopic suction device in accordance with one embodiment of the present invention;

FIG. 4 is a cross-sectional view of the endoscopic apparatus taken along line 4-4 of FIG. 3;

FIG. 5 is an environmental view of the assembly applying suction on a lesion during mucosectomy; and

FIG. 6 is an environmental view of an exposed muscularis propria layer after snare excision.

### DETAILED DESCRIPTION OF THE INVENTION

Embodiments of the present invention provide an endoscopic suction device for an endoscope during mucosectomy. The device is a distal cap that is placed directly over the distal end of an endoscope. The distal cap includes a side or lateral opening to suction mucosa into its chamber so that the mucosa can be resected using a snare. In one embodiment, the device or distal cap comprises a connecting base attachable to the endoscope and a distal tip extending from the base. The tip has a lateral suction opening formed through the side in fluid communication with a suction chamber to receive a lesion during mucosectomy.

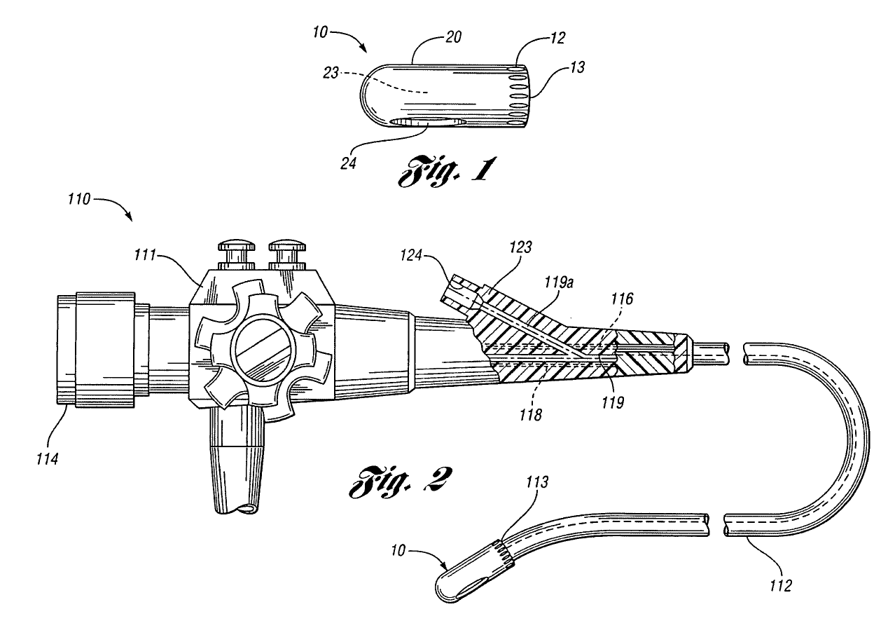

FIG. 1 illustrates an endoscopic suction device or distal cap 10 comprising a connecting base 12 having an open end 13 attachable to the insertion tube of an endoscope. The open end 13 may be attached to the insertion tube by any suitable means, e.g., threaded connection, press fit, or bonded attachment. As shown, the endoscopic suction device 10 further comprises a distal tip 20 integrally extending distally from the connecting base 12. In this embodiment, the distal tip 20 comprises a suction chamber 23 formed therein and in fluid communication with the open end 13. The suction chamber is able to hold a lesion to be removed during mucosectomy. Preferably, the suction chamber has a lateral suction opening 24 formed through the side of the device 10 for suctioning a lesion to be held in the suction chamber 23 during mucosectomy. The lateral suction opening 24 is in fluid communication with the open end 13 so that a vacuum or suction source may be used. Preferably, the distal cap 10 may have any suitable length, e.g., between about 1.5 and 4 centimeters. Furthermore, although the drawings depict the distal cap 10 having an elongate or bullet shaped tip, the distal cap may take on any other suitable shape.

As shown, the suction opening 24 is formed laterally through the distal tip 20. This allows the suction opening 24 to be more easily disposed over the lesion, thereby being removed more conveniently as will be described in greater detail below. Preferably, the suction opening 24 may have an oval or a generally circular shape; however, the suction opening 24 may take on any other shape without falling beyond the scope or spirit of the present invention. Moreover, the area adjacent the suction opening 24 may be planar or flat to increase contact with the mucosal tissue and create a seal for enhanced suctioning. With a transparent distal tip 20 along with the laterally formed suction opening 24, the physician is able to more clearly maneuver or manipulate the endoscopic parts to perform the mucosectomy procedure. In one embodiment, the suction opening 24 is configured to fit over mucosa for a mucosectomy treatment. Alternatively, the suction opening 24 may be relatively smaller in area to fit over the lesion, but with a suction source that effectively suctions the lesion in the chamber for mucosectomy.

The endoscopic suction device 10 may be made of any suitable material, preferably transparent material. In one embodiment, at least a portion of the endoscopic device could be made of metal, metal alloy, or an opaque material. However, it is advantageous for the device 10 to be made of transparent material. For example, the endoscopic suction device 10 may comprise one of super elastic material, polycarbonate plastic, nitinol, cobalt-chromium-nickel-molybdenum-iron alloy, or cobalt-chrome alloy, polytetrafluoroethylene (PTFE), polyethylene, polypropylene, perfluoroelastomer, fluoroelastomer, nitrile, neoprene, polyurethane, silicone, polytetrafluroethylene, styrene-butadiene, rubber, or polyisobutylene.

In this embodiment, the endoscopic suction device is preferably configured to be able to receive a snare disposed through the open end and situated adjacent the lateral suction opening for receiving mucosa or a lesion during a mucosectomy procedure. Thus, in use, a lesion is suctioned through the opening 24 and received within a loop of the snare for resection or removal during mucosectomy.

FIG. 2 illustrates a flexible endoscopic apparatus or instrument 110 comprising the endoscopic suction device 10 in accordance with one embodiment of the present invention. The apparatus 110 has a length that permits access to the deeper regions of a hollow body organ. In certain embodiments, the flexible apparatus 110 can be sized for insertion into the alimentary tract. In accordance with one embodiment, the apparatus 110 includes a conventional endoscope with an operating control section 111 and a flexible section 112 that terminates at a distal insertion end 113. The operating control section 111 includes a viewing end 114 remote from the insertion end 113, through which a ligating procedure can be directly observed.

It is to be understood that any other suitable endoscopic apparatus may be used with the ligator assembly described above. For example, various endoscopic ligating apparatus may be used including but not limited to U.S. Pat. No. 6,007,551 entitled “Endoscopic Ligating Apparatus” filed on Sep. 6, 1996 and U.S. Pat. No. 5,624,453 entitled “Endoscopic Ligating Instrument” filed on Oct. 30, 1995, the entire contents of each are incorporated herein by reference.

Referring to FIGS. 2 and 4, the endoscopic instrument 110 may include a plurality of channels extending from the operating control section 111 and through the flexible section 112 to the insertion end 113. For example, the instrument 110 can include an illumination channel 116 through which a fiberoptic cable is inserted for the transmission of light from a light source. A viewing channel 117 can also be provided with a fiberoptic cable for viewing purposes, while a third channel 118 can be provided for application of suction at the surgical site. The endoscopic instrument 110 can also include a working channel 119 through which a plurality of tools and instruments can be extended such as a polypectomy snare for resection of a lesion, an irrigation channel 120 to allow delivery of fluid to the ligation site, and an air channel 121 that can be used to deliver pressurized air, such as for cleaning the lens at the insertion end of the viewing channel 117.

In one embodiment, the endoscopic instrument 110 also includes an auxiliary port portion 123 having a proximal opening 124. The working channel 119 extends into the auxiliary port 123 by way of a working channel extension 119a. Each of the channels preferably opens at the distal or insertion end 113 of the flexible section 112 of the endoscopic instrument 110.

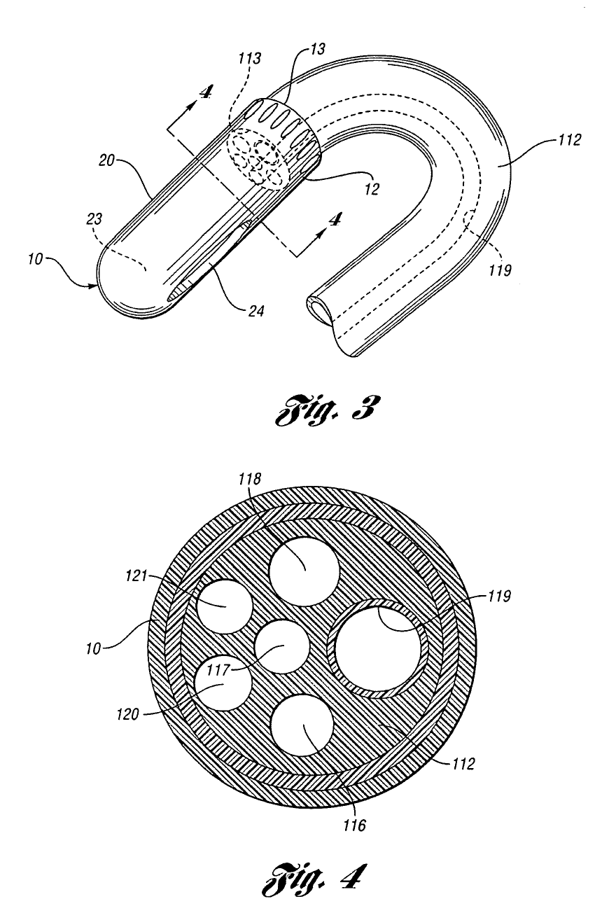

The endoscope forming part of the instrument 110 of FIG. 3 can be of many different types. For example, the endoscope can be of the type commercially provided by Olympus, Pentax, or Fujinon. While most of the working components of these endoscopes are similar, each may have a different configuration for the proximal opening 124 and the auxiliary port 123. Each of these specifically identified endoscopes, and other commercially available endoscopes, utilize different sealing members (not shown) at the proximal opening 124 of the auxiliary port 123. It is understood that the various aspects of the present invention accommodate the secure attachment to various configurations and dimensions of a variety of endoscopes.

Referring now to FIG. 4, details of the endoscopic suction device 10 can be seen. In this embodiment, the device 10 is disposed at the insertion end 113 of the flexible section 112 of the endoscope. The material of the device 10 should be sufficiently strong or rigid to receive lesions to be suctioned therein and resected by a polypectomy snare. The device 10 is preferably removably mountable to the insertion end 113 of the flexible endoscope section 112.

In use, the endoscopic suction device is preferably provided separately from the endoscopic instrument. At an appropriate time in the use of the flexible endoscopic apparatus, the device 10 can be mounted about the cylindrical surface of the flexible endoscope section by any suitable means such as by cooperating threads.

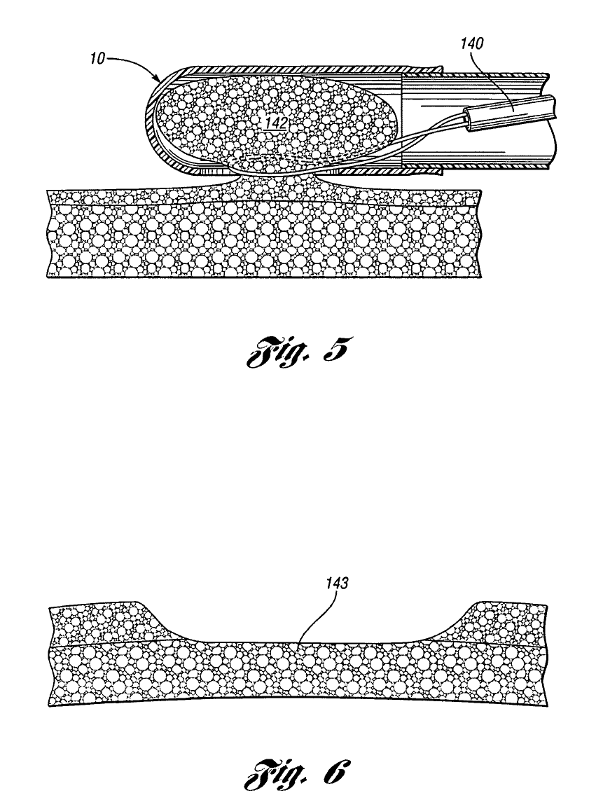

FIGS. 5 and 6 illustrate a method of mucosectomy in accordance with one example of the present invention. In this example, the technique of mucosectomy uses a braided snare. In another example, a monofilament stiff-wire polypectomy snare may be used. As shown in FIG. 5, the polypectomy snare is disposed distally through a working channel of the endoscope. The snare is then moved through the distal end of the endoscope and is opened for positioning relative to a lesion to be removed during mucosectomy. Upon suctioning proximally through the distal tip, the polypectomy snare is opened and placed against the mucosal surface about the lesion. The snare is then relatively slowly moved towards a closed position, thereby reducing blood flow through the lesion. In another example, the snare may also be introduced outside of the distal tip prior to suctioning through the distal tip.

FIG. 5 illustrates the polypectomy snare 140 in a relatively closed position around the neck of the lesion 142. In this example, once tightened around the lesion, the endoscopic suction device 10 is positioned about the lesion for suctioning. More specifically, the lesion 142 is disposed through the suction opening 24 into the suction chamber 23 of the distal tip 20 of the endoscopic suction device 110. The device 10 maintains a suitable vacuum to maintain the lesion within the suction chamber 23 of the device 10. The snare 140 is then further tightened around the lesion 142 and lifted away from its vessel wall. Pure coagulation current as known may be applied to transect the lesion 142. Lifting the lesion 142 further into the chamber 23 as current is applied helps further prevent transmural extension of the burn.

FIGS. 5 and 6 illustrate a muscularis propria layer 143. In this example, the muscularis propria layer 143 is preferably exposed after the snare excision. This indicates that a complete mucosectomy has been performed. The lesion 142 is received in the suction chamber 23 of the device 10 as a vacuum is maintained therethrough. The device 10 then may be retracted through the suction channel and out of the system for disposal or retainment of the lesion.

While the present invention has been described in terms of preferred embodiments, it will be understood, of course, that the invention is not limited thereto since modifications may be made to those skilled in the art, particularly in light of the foregoing teachings.

1. An endoscopic suction device of a distal end of an endoscope for mucosectomy, the device comprising:
a connecting base having an open end attachable to the distal end of the endoscope; and
a distal tip extending from the connecting base and having a closed distal end, the distal tip comprising a suction chamber formed therein and in fluid communication with the open end, the suction chamber having a suction opening formed laterally therethrough for suctioning lesions during mucosectomy.

2. The device of claim 1 wherein the connecting base is configured to be in fluid communication with the endoscope when attached thereto.

3. The device of claim 1 wherein the distal tip is integrally connected with the connecting base.

4. The device of claim 1 wherein the device comprises transparent polymeric material.

5. The device of claim 1 wherein the suction opening is formed longitudinally through the distal tip.

6. An endoscope apparatus having a suction apparatus, the apparatus comprising:
an endoscopic assembly for endoscopy, the assembly comprising:
an insertion tube, the insertion tube having a plurality of channels through which endoscopic parts are disposed; and
a control system in mechanical and fluid communication with the insertion tube, the control system being configured to control at least one of the endoscopic parts; and
an endoscopic suction device of a distal end of the endoscope, the endoscopic suction device comprising:
a connecting base having an open end attachable to the distal end of the endoscope; and
a distal tip extending from the connecting base and having a closed distal end, the distal tip comprising a suction chamber formed therein and in fluid communication with the open end, the suction chamber having a suction opening formed laterally therethrough for suctioning lesions during mucosectomy.

7. The apparatus of claim 6 wherein the connecting base is configured to be in fluid communication with the endoscope when attached thereto.

8. The apparatus of claim 6 wherein the distal tip is removably connected to the connecting base.

9. The apparatus of claim 6 wherein the device comprises one of metal and polymeric material.

10. The apparatus of claim 6 wherein the suction opening is formed longitudinally through the distal tip.

11. The apparatus of claim 6 wherein the distal tip comprises a distal end, the suction opening being formed through the distal end of the distal tip.

12. The apparatus of claim 6 further comprising a snare disposed through the open end and adjacent the suction opening for receiving a lesion during mucosectomy.

13. A method of mucosectomy of mucosal tissue of a patient, the method comprising:
disposing a polypectomy snare distally through a working channel of the endoscope having a distal end;
advancing the snare through the distal end to an endoscopic suction device attached to the distal end, the device comprising a connecting base and a distal tip extending from the connecting base, the connecting base having an open end attached to the distal end of the endoscope, the distal tip having a closed distal end, the distal tip comprising a suction chamber formed therein and in fluid communication with the open end, the suction chamber having a suction opening formed laterally therethrough for suctioning lesions during mucosectomy;
opening the snare adjacent the suction opening to receive the mucosal tissue;
suctioning the mucosal tissue through the suction opening; and
receiving the mucosal tissue with the snare.

14. The method of claim 13 wherein the connecting base is configured to be in fluid communication with the endoscope when attached thereto.

15. The method of claim 13 wherein the distal tip is integrally connected with the connecting base.

16. The method of claim 13 wherein the device comprises transparent polymeric material.

17. The method of claim 13 wherein the suction opening is formed longitudinally through the distal tip.
